(19) United States
(12) Patent Application Publication
(10) Pub. No.: US 2024/0299750 A1
(43) Pub. Date: Sep. 12, 2024

(54) SYSTEMS AND METHODS FOR FIELD OPTIMIZATION FOR TARGETED NEURAL STIUMATION

(71) Applicant: REGENTS OF THE UNIVERSITY OF MICHIGAN, Ann Arbor, MI (US)

(72) Inventors: Ehsan Mirzakhalili, Philadelphia, PA (US); Scott Lempka, Ann Arbor, MI (US)

(21) Appl. No.: 18/597,657

(22) Filed: Mar. 6, 2024

Related U.S. Application Data
(60) Provisional application No. 63/450,531, filed on Mar. 7, 2023.

(51) Int. Cl. A61N 1/36; G05B 15/02

(52) U.S. Cl. CPC A61N 1/36157; G05B 15/02; A61N 1/36071

(57) ABSTRACT

Field optimization techniques for targeted neural stimulation are provided using finite element method (FEM) model of electrodes and of target region of interest (ROI) biological features. The FEM model estimates the electric potential fields generated by applied stimulation, and include single or multiple electrode configurations and further include biological features, such as encapsulation, dorsal rootles, dura mater, and the vertebral column, in various examples of spinal cord stimulation. The techniques use a single- or multiple-factor optimization that maximize stimulation to the ROI while minimizing effects on a region of avoidance (ROA). Various configurations apply a generalized Lagrange multiplier method to formulate the optimization problem and an epsilon-constraint method to find the Pareto front for multi-objective optimization problems.

### CROSS-REFERENCE TO RELATED APPLICATIONS

This application claims the benefit of U.S. Provisional Application No. 63/450,531, filed Mar. 7, 2023, the entirety of which is hereby incorporated herein by reference.

### STATEMENT OF GOVERNMENT SUPPORT

This invention was made with government support under OD028191 awarded by the National Institutes of Health. The government has certain rights in the invention.

### BACKGROUND

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

Spinal cord stimulation (SCS) is a common therapy to treat chronic pain. Typical SCS therapies use electrodes to treat a patient. Therapy outcomes depend heavily on stimulation parameters, including the stimulation configuration of the electrodes (i.e., combination of anodes and cathodes). Theoretically, with proper stimulation configurations, it is possible to maximize the clinical benefits of SCS by activating neural tissues in a region of interest (ROI), while minimizing activation in a region of avoidance (ROA) that can be associated with side effects. For example, some have proposed stimulation configurations that directly target neurons in the dorsal horn without activating axons in the dorsal column.

These conventional stimulation configurations for SCS suffer from various shortcomings. There is often a high rate of failure to provide pain relief. Uncomfortable sensations often result in regions related or/and unrelated to the area of pain. The efficacy of pain relief diminishes over time. Of course, SCS may fail in some patients because of the inability to provide targeted stimulation, for example, due to poor placement of the electrodes or electrode migration. Plus, there is a therapeutic window that one must operate within. One cannot just turn the stimulation up until the target areas are stimulated, because the stimulation may then begin to activate off-target areas that lead to side effects.

SCS techniques are also limited due to the complex nature of signals and signal interactions involved in pain expression and in pain abatement. As a computational endeavor, researchers in the field are presented with a parameter space for these stimulation systems that is very large. It is not possible to explore these parameters spaces within standard clinical visits. Therefore, computational models provide a valuable clinical decision support tool. However, conventional SCS computational models are limited in parameter space size. They are typically based on one or no more than a handful of model parameters to allow for optimizations. And in this way, they are quite rigid in approach and do not allow for optimizing stimulation signals at precise locations. They do not allow for optimizing stimulations at single points, over two-dimensional regions, or over three-dimensional regions that avoids the foregoing problems, such as off-target activation. Further, conventional computational model techniques are field specific and do not allow for optimizing stimulation signals across different types of fields, different types of modalities.

There is a need for techniques for optimized, targeted neural stimulation.

### SUMMARY OF THE INVENTION

The present application describes systems and methods of stimulation configurations that deploy computational models in developing an optimization framework for selective neural activation in regions of interest, such as the spinal cord, brain, peripheral nerves, or cardiac tissue. The present application can overcome the limitations of conventional systems, offering, for example, flexibility in optimizing stimulation signals. Optimization frameworks can be designed in various forms, allowing for optimization of target region stimulation, at a single point as well as over one-dimensional (1D), two-dimensional (2D), and three-dimensional (3D) domains. Further, optimization frameworks are not field dependent, but rather, the present techniques can optimize stimulation signals for any number of different signal types, such as an electric field (negative gradient of the electric potential), an activating function (second derivative of the electric potential), or even electric potential. Furthermore, the stimulation signals can be optimized in any direction. Of usefulness, the present techniques provide for exact optimizations, as opposed to methods that would seek to search for optimum configurations.

In various examples, the present application describes techniques using a finite element method (FEM) model of electrodes and of target region of interest (ROI) biological features. The FEM model is used to estimate the electric potential fields generated by stimulation applied through different electrode configurations. These FEM models may include single or multiple electrode configurations, for example, with the FEM model further including biological features, such as encapsulation, dorsal rootles, dura mater, and the vertebral column, in an example of spinal cord stimulation (SCS). In various examples, the present application relies upon a single- or multiple-factor optimization, by maximizing stimulation to the ROI while minimizing effects on a region of avoidance (ROA) (off-target region). For example, various configurations apply a generalized Lagrange multiplier method to formulate the optimization problem and an epsilon-constraint method to find the Pareto front for multi-objective optimization problems.

The present techniques can be applied in various clinical settings, with some examples herein describing the use of novel computational models to develop an optimization framework for investigating and affecting selective neural activation in the spinal cord.

In an aspect, a method for controlling targeted neural stimulation to a subject for treatment, the method comprising: determining, at one or more processors, a stimulation signal for each of a plurality of electrodes positioned for treatment of a region of interest, using a discretized point model representative of at least the region of interest; determining, at the one or more processors, a stimulation superposition signal using the stimulation signals for each electrode, the stimulation superposition signal being determined for each discretized point in the discretized point model and from constraining all current fractions of the plurality of electrodes according to a maximum bound and/or a minimum bound; from the determined stimulation signal superposition signal, performing, at the one or more processors, an optimization on the stimulation signal data to maximize a superposition field at the region of interest according to an objective function, wherein performing the optimization includes, applying a smooth maximum operator to the objective function to form a differentiable objective function, and applying a Lagrange multiplier to obtain the set of expressions that maximizes the objective function and satisfies the constraints on the current fractions, obtaining from the set of expressions, discrete points of the Lagrangian function corresponding to a maximum, a minimum, or a none value, and identifying, from the optimization, a stimulation signal combination for controlling each of the plurality of electrodes for treatment of the subject and corresponding to the optimization of the superposition field according to the objective function; and providing control signals to the plurality of electrodes and based on the stimulation signal parameters to affect treatment of the region of interest by the plurality of electrodes.

In another aspect, a system for controlling targeted neural stimulation parameters for treatment of a subject, the system comprising: one or more processors; and one or more memories having stored thereon computer-executable instructions that, when executed, cause the computing system to: determine, at one or more processors, a stimulation signal for each of a plurality of electrodes positioned for treatment of a region of interest, using a discretized point model representative of at least the region of interest; determine, at the one or more processors, a stimulation superposition signal using the stimulation signals for each electrode, the stimulation superposition signal being determined for each discretized point in the discretized point model and from constraining all current fractions of the plurality of electrodes according to a maximum bound and/or a minimum bound; from the determined stimulation signal superposition signal, perform, at the one or more processors, an optimization on the stimulation signal data to maximize a superposition field at the region of interest according to an objective function, wherein performing the optimization includes instructions that, when executed, cause the computing system to, apply a smooth maximum operator to the objective function to form a differentiable objective function, apply a Lagrange multiplier to obtain the set of expressions that maximizes the objective function and satisfies the constraints on the current fractions, obtain from the set of expressions, discrete points of the Lagrangian function corresponding to a maximum, a minimum, or a none value, and identify, from the optimization, a stimulation signal combination for controlling each of the plurality of electrodes for treatment of the subject and corresponding to the optimization of the superposition field according to the objective function; and provide control signals to the plurality of electrodes and based on the stimulation signal parameters to affect treatment of the region of interest by the plurality of electrodes.

In yet another aspect, a non-transitory computer-readable storage medium storing executable instructions that, when executed by a processor, cause a computer to: determine, at one or more processors, a stimulation signal for each of a plurality of electrodes positioned for treatment of a region of interest, using a discretized point model representative of at least the region of interest; determine, at the one or more processors, a stimulation superposition signal using the stimulation signals for each electrode, the stimulation superposition signal being determined for each discretized point in the discretized point model and from constraining all current fractions of the plurality of electrodes according to a maximum bound and/or a minimum bound; from the determined stimulation signal superposition signal, perform, at the one or more processors, an optimization on the stimulation signal data to maximize a superposition field at the region of interest according to an objective function, wherein performing the optimization includes instructions that, when executed, cause the computing system to, apply a smooth maximum operator to the objective function to form a differentiable objective function, and apply a Lagrange multiplier to obtain the set of expressions that maximizes the objective function and satisfies the constraints on the current fractions, obtain from the set of expressions, discrete points of the Lagrangian function corresponding to a maximum, a minimum, or a none value, and identify, from the optimization, a stimulation signal combination for controlling each of the plurality of electrodes for treatment of the subject and corresponding to the optimization of the superposition field according to the objective function; and provide control signals to the plurality of electrodes and based on the stimulation signal parameters to affect treatment of the region of interest by the plurality of electrodes.

In yet another aspect, a method for controlling targeted neural stimulation to a subject for treatment, the method comprising: determining, at one or more processors, a stimulation signal for each of a plurality of electrodes positioned for affecting a region of interest and for affecting a region of avoidance, using a discretized point model representative of the region of interest and of the region of avoidance; determining, at the one or more processors, a stimulation superposition signal using the stimulation signals for each electrode, the stimulation superposition signal being determined for each discretized point in the region of interest and in the region of avoidance and constraining all current fractions of the plurality of electrodes according to a maximum bound and/or a minimum bound; from the determined stimulation signal superposition, performing, at the one or more processors, a multi-objective optimization on the stimulation signal data to maximize a superposition field at the region of interest and to minimize a superposition field at the region of avoidance according to a multi-objective function, wherein performing the optimization includes, applying a smooth maximum operator to the objective function to form a differentiable objective function, and applying a Lagrange multiplier to obtain the set of expressions that maximizes the objective function and satisfies the constraints on the current fractions, and obtaining from the set of expressions, discrete points of the Lagrangian function corresponding to a maximum, a minimum, or a none value, using an epsilon-constraint method to construct a Pareto front of solutions for the multi-objective function, selecting an objective function of the multi-objective function as a primary objective and converting the other objective functions to inequality constraints, applying a generalized Lagrange multiplier to obtain the set of expressions that satisfies the multi-objective function and satisfies the constraints on the current fractions; and identifying, from the optimization, a stimulation signal combination for controlling each of the plurality of electrodes for treatment of the subject and corresponding to the optimization of the superposition field according to the objective function; and providing control signals to the plurality of electrodes and based on the stimulation signal parameters to affect treatment of the region of interest and of the region of avoidance by the plurality of electrodes.

In yet another aspect, a system controlling targeted neural stimulation to a subject for treatment, the system comprising: one or more processors; and one or more memories having stored thereon computer-executable instructions that, when executed, cause the computing system to: determine, at one or more processors, a stimulation signal for each of a plurality of electrodes positioned for affecting a region of interest and for affecting a region of avoidance, using a discretized point model representative of the region of interest and of the region of avoidance; determine, at the one or more processors, a stimulation superposition signal using the stimulation signals for each electrode, the stimulation superposition signal being determined for each discretized point in the region of interest and in the region of avoidance and constraining all current fractions of the plurality of electrodes according to a maximum bound and/or a minimum bound; from the determined stimulation signal superposition, perform, at the one or more processors, a multi-objective optimization on the stimulation signal data to maximize a superposition field at the region of interest and to minimize a superposition field at the region of avoidance according to a multi-objective function, wherein the instructions to perform the optimization include instructions to, apply a smooth maximum operator to the objective function to form a differentiable objective function, apply a Lagrange multiplier to obtain the set of expressions that maximizes the objective function and satisfies the constraints on the current fractions, obtain from the set of expressions, discrete points of the Lagrangian function corresponding to a maximum, a minimum, or a none value, use an epsilon-constraint method to construct a Pareto front of solutions for the multi-objective function, select an objective function of the multi-objective function as a primary objective and converting the other objective functions to inequality constraints, apply a generalized Lagrange multiplier to obtain the set of expressions that satisfies the multi-objective function and satisfies the constraints on the current fractions; and identify, from the optimization, a stimulation signal combination for controlling each of the plurality of electrodes for treatment of the subject and corresponding to the optimization of the superposition field according to the objective function; and provide control signals to the plurality of electrodes and based on the stimulation signal parameters to affect treatment of the region of interest and of the region of avoidance by the plurality of electrodes.

In yet another aspect, a non-transitory computer-readable storage medium storing executable instructions that, when executed by a processor, cause a computer to: determine, at one or more processors, a stimulation signal for each of a plurality of electrodes positioned for affecting a region of interest and for affecting a region of avoidance, using a discretized point model representative of the region of interest and of the region of avoidance; determine, at the one or more processors, a stimulation superposition signal using the stimulation signals for each electrode, the stimulation superposition signal being determined for each discretized point in the region of interest and in the region of avoidance and constraining all current fractions of the plurality of electrodes according to a maximum bound and/or a minimum bound; from the determined stimulation signal superposition, perform, at the one or more processors, a multi-objective optimization on the stimulation signal data to maximize a superposition field at the region of interest and to minimize a superposition field at the region of avoidance according to a multi-objective function, wherein the instructions to perform the optimization include instructions to, apply a smooth maximum operator to the objective function to form a differentiable objective function, apply a Lagrange multiplier to obtain the set of expressions that maximizes the objective function and satisfies the constraints on the current fractions, obtain from the set of expressions, discrete points of the Lagrangian function corresponding to a maximum, a minimum, or a none value, use an epsilon-constraint method to construct a Pareto front of solutions for the multi-objective function, select an objective function of the multi-objective function as a primary objective and converting the other objective functions to inequality constraints, apply a generalized Lagrange multiplier to obtain the set of expressions that satisfies the multi-objective function and satisfies the constraints on the current fractions; and identify, from the optimization, a stimulation signal combination for controlling each of the plurality of electrodes for treatment of the subject and corresponding to the optimization of the superposition field according to the objective function; and provide control signals to the plurality of electrodes and based on the stimulation signal parameters to affect treatment of the region of interest and of the region of avoidance by the plurality of electrodes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 is a schematic diagram of a system for controlling targeted neural stimulation to a subject for treatment, in accordance with an example.

FIG. 2A illustrates a portion of a spinal cord that may be modeled using a discretized point model, in accordance with an example. FIG. 2B illustrates an example of a portion of the total electric potential field that results from a stimulation configuration generated using techniques herein, in accordance with an example.

FIG. 3 illustrates an example single-objective optimization module, as may be used in the system of claim 1, in accordance with an example.

FIG. 4 illustrates a process for single-objective optimization as may be performed by the module of FIG. 3, in accordance with an example.

FIG. 5 illustrates a process for a canonical approach for solving a finite element method model as may be performed by the process of FIG. 4, in accordance with an example.

FIG. 6 illustrates a process for a patient-specific approach for solving a finite element method model as may be performed by the process of FIG. 4, in accordance with an example.

FIG. 7 illustrates a process for a single-objective optimization as may be performed by the process of FIG. 4, in accordance with an example.

FIG. 8 illustrates a process for a single-objective solver as may be performed by the process of FIG. 4, in accordance with an example.

FIG. 9 illustrates an example one-dimension region of interest and resulting stimulation configuration, applying a single-objective optimization, in accordance with an example.

FIG. 10 illustrates an example three-dimension region of interest and resulting stimulation configuration, applying a single-objective optimization, in accordance with an example.

FIG. 11 illustrates an example multi-objective optimization module, as may be used in the system of claim 1, in accordance with an example.

FIG. 12 illustrates a process for multi-objective optimization as may be performed by the module of FIG. 11, in accordance with an example.

FIG. 13 illustrates a process for a multi-objective optimization as may be performed by the process of FIG. 11, in accordance with an example.

FIG. 14 illustrates a process for a multi-objective solver as may be performed by the process of FIG. 11, in accordance with an example.

FIG. 15 illustrates an example one-dimension region of interest and a one-dimensional region of avoidance and resulting stimulation configuration, applying a multi-objective optimization, in accordance with an example.

FIG. 16 illustrates an example three-dimension region of interest and a three-dimensional region of avoidance and resulting stimulation configuration, applying a multi-objective optimization, in accordance with an example.

FIG. 17 illustrates an example Pareto front plot for two different objectives, Objective 1 and Objective 2, and all the four different combinations of solutions for the two (Objective 1| Objective 2): MIN| MAX, MIN| MIN, MAX| MAX, and MAX| MIN.

FIG. 18 illustrates an example of a multi-objective optimization modules use of an epsilon-constraint method to create the Pareto fronts, in accordance with an example.

### DETAILED DESCRIPTION

The present application describes systems and methods with stimulation configurations with computational models used to develop an optimization framework for selective neural activation, such as in the spinal cord, deep brain, or cardiac tissue. The present application describes techniques using a finite element method (FEM) model of electrodes and target region of interest (ROI) biological features, and the FEM model is used to estimate the electric potential fields generated by stimulation applied through different electrode configurations. For example, FEM models may include single or multiple electrode configurations, with the FEM model further including biological features, such as encapsulation, dorsal roots, dura mater, and the vertebral column, in an example of spinal cord stimulation (SCS). The present application relies upon a single- or multiple-factor optimization to optimize stimulation, by maximizing stimulation to the ROI while minimizing effects on a region of avoidance (ROA). For example, the present application in some configurations uses a generalized Lagrange multiplier method to formulate an optimization problem and an epsilon-constraint method to find the Pareto front for multi-objective optimization problems. Optimization problems are expressions or conditions for maximizing a desired field in a selected ROI (e.g., single-objective optimization) or in multiple ROIs (e.g., multi-objective optimization). The result of determining solutions to an optimization problem is a combination of electrode stimulation signals for affecting treatment. Further, the application of these electrode stimulation signals may be visualized or the field in the ROI(s) and the lack of field elsewhere can be visualized using stored FEM solutions herein.

The present techniques can overcome deficiencies in the state of the art. The present techniques can generate an electrode configuration that maximizes the field that is presumed by the operator to provide clinical benefits in the selected region in case of the single-objective optimization. In the case of multi-objective optimization, the present techniques can generate several different optimization solutions each with trade-offs between targeting ROIs and avoiding ROAs, allowing an SCS operator or clinician to select electrode configurations based on the desired balancing of those trade-offs. Indeed, one of the advantages of the present techniques is that an operator can specifically select areas or volumes with respect to ROIs and ROAs, whereas conventional systems only allow users to specify points to focus the stimulation or, with limited efficacy, a general 2D region. Furthermore, the present techniques need not rely on a searching operation which can be quite time consuming, but rather may be built upon solving a series of nonlinear equations to find optimum configurations provided that they exist.

More specially, in some examples, the present techniques are used to show that terminal and axonal stimulation are governed by first and second derivatives of the electric potential field, respectively. From that, the present techniques can maximize the respective fields in ROIs while minimizing the respective fields in ROAs. While stimulation configurations that maximize the objective function in the ROIs (i.e., lowest activation thresholds for the ROIs) can also produce neural activation in the ROAs, with the present techniques it is possible to avoid/minimize activation in the ROAs by using specific stimulation configurations that require higher stimulation amplitudes to activate the ROIs. Indeed, this balance between activation thresholds in the ROIs and level of activity in the ROAs is a design parameter that can be used to create Pareto fronts in the multi-objective optimization.

As a result, the present techniques can provide systems and methods with an optimization framework that finds stimulation configurations that target specific neural structures for axonal and/or terminal stimulation.

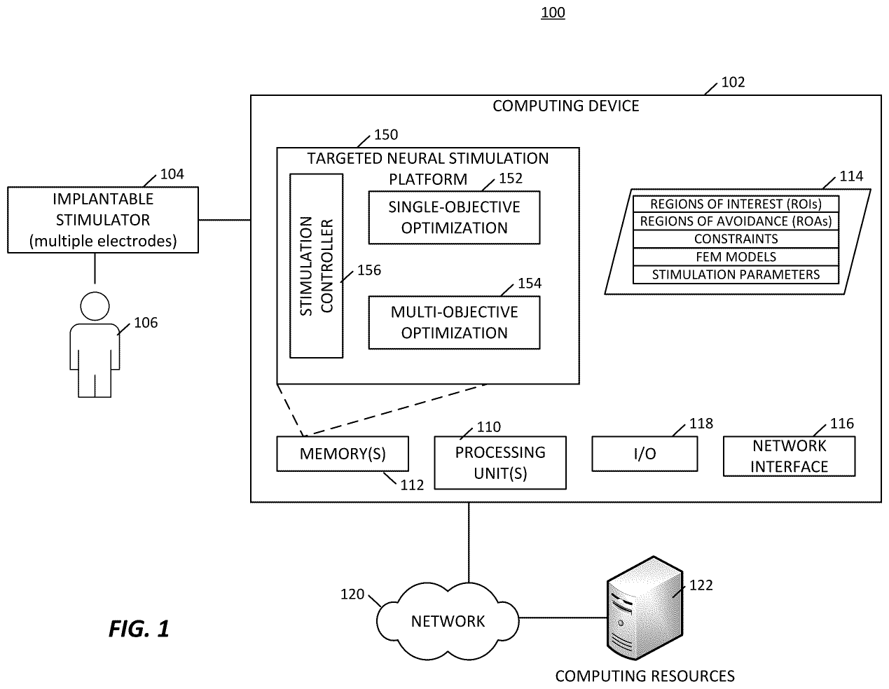

FIG. 1 illustrates an example targeted neural stimulation environment 100 for implementing the processes and methods described herein. The environment 100 includes a computing device 102 configured to develop stimulation configurations and for controlling application of target stimulation through an implantable stimulator 104 treating a patient 106. In other examples, the stimulators herein may be external stimulators. Examples include a stimulator worn on the skin or otherwise placeable on or near the skin and communicatively coupled to implanted stimulation electrodes. In some examples, the stimulators may be electrodes placed on the surface of the skin to apply transcutaneous stimulation. Thus, examples herein to implantable stimulator should be considered as applying to fully- or partially-external stimulators, as well. Further, references to stimulator herein refers to electrodes and may in various examples include the hardware (e.g., electronics, battery, etc.) or pulse generator that generate the pulses/waveforms. For example, a partially-external stimulator may be one with implanted electrodes and an external communicatively coupled pulse waveform generator. The environment 100 and processes and methods herein may be used to provide various types of stimulations, including as described in various examples, targeted neural stimulation of the dorsal column, dorsal horn, the brain, the peripheral nervous system, the autonomic nervous system, etc. The present techniques may be extended to other types of stimulations altogether, including cardiac stimulation, deep brain stimulation (DBS), etc.

In the illustrated example, the computing device 102 includes a includes one or more processing units 110, a local data storage 112, a computer-readable memory 114, a network interface 116, and Input/Output (I/O) interfaces 118 connecting the computing device 102 to a display (not shown) and user input device (not shown).

The computing device 102 may be implemented on a single computer processing device or multiple computer processing devices. The computing device 102 may be implemented on a network accessible computer processing device, such as a server, or implemented across distributed devices connected to one another through a communication link. In other examples, functionality of the computing device 102 may be distributed across any number of devices, including the portable personal computer, smart phone, electronic document, tablet, and desktop personal computer devices shown. In other examples, the functionality of the computing device 102 may be cloud based, such as, for example one or more connected cloud CPU (s) customized to perform machine learning processes and computational techniques herein.

In the illustrated example, the network interface 116 is connected to a network 120 which may be a public network, such as the Internet, private network such as research institution's or corporation's private network, or any combination thereof. Networks can include, local area network (LAN), wide area network (WAN), cellular, satellite, or other network infrastructure, whether wireless or wired. The network can utilize communications protocols, including packet-based and/or datagram-based protocols, such as internet protocol (IP), transmission control protocol (TCP), user datagram protocol (UDP), or other types of protocols. Moreover, the network 120 can include a number of devices that facilitate network communications and/or form a hardware basis for the networks, such as switches, routers, gateways, access points (such as a wireless access point as shown), firewalls, base stations, repeaters, backbone devices, etc. In the illustrated example, the computing device 102 is connected to a computing resource 122 through the network 120.

The memory 112 may be a computer-readable media and may include executable computer-readable code stored thereon for programming a computer (e.g., comprising a processor(s) and GPU(s)) to the techniques herein. Examples of such computer-readable storage media include a hard disk, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units 110 of the computing device 102 may represent one or more CPU-type processing units, one or more GPU-type processing units, one or more a field-programmable gate arrays (FPGA), another class of digital signal processor (DSP), or other hardware logic components that can be driven by a CPU.

In the illustrated example, in addition to storing an operating system, the memory 112 stores a targeted neural stimulation platform 150, configured to execute various processes described and illustrated herein. In an example, the targeted neural stimulation platform 150 is configured to control targeted neural stimulation under single optimization or multi-objective optimization processes described herein. The targeted neural stimulation platform 150 may be configured to output a targeted stimulation protocol defining control signals to be provided to electrodes to affect treatment of a ROI.

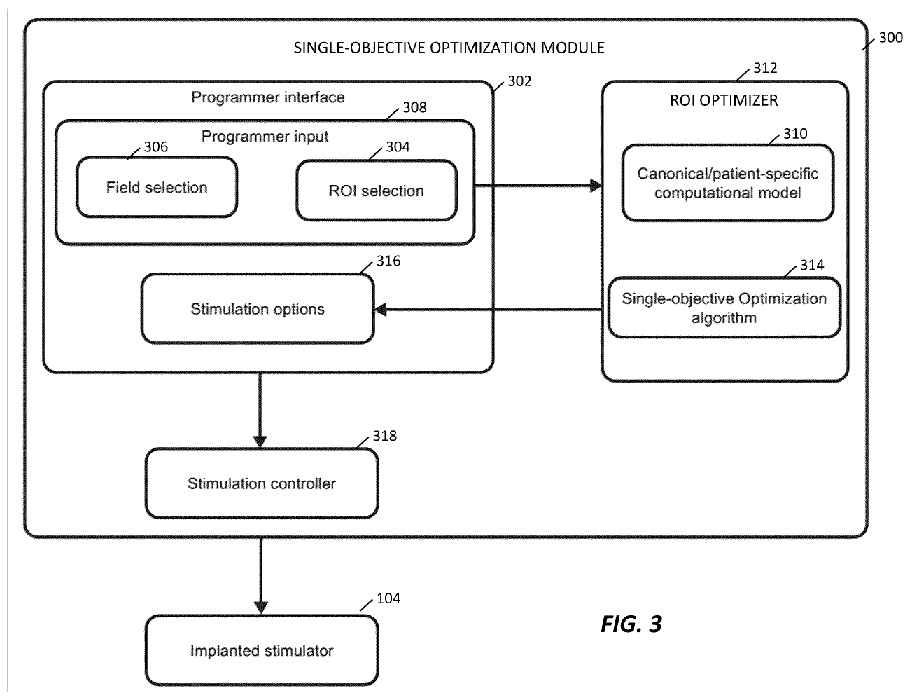

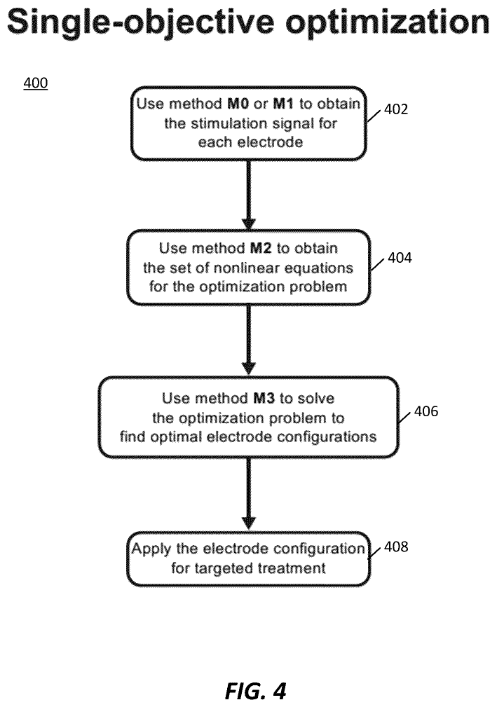

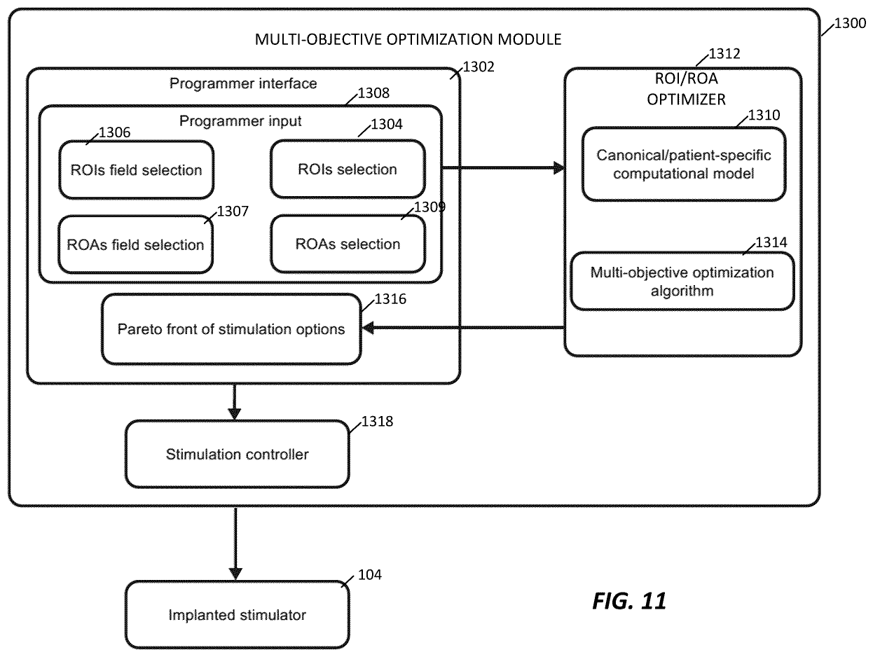

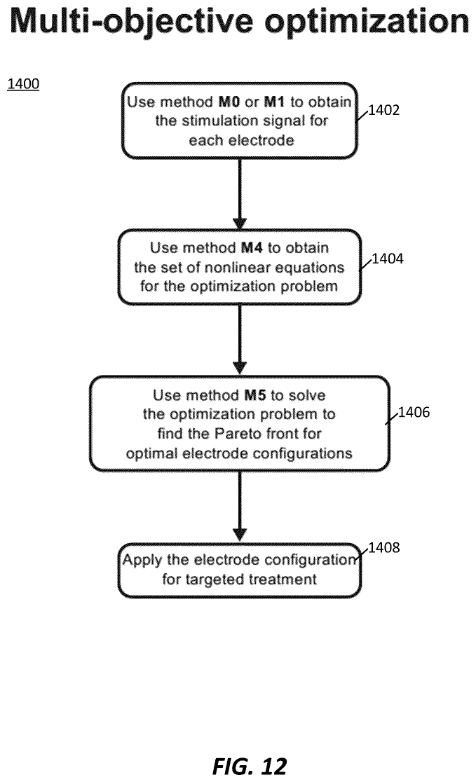

In the illustrated example, the targeted neural stimulation platform 150 includes a single-objective optimization module 152 and a multi-objective optimization module 154, each configured to provide stimulation configuration data to a stimulation controller 156 that controls operation of the implantable stimulator 104 to affect targeted stimulation to a ROI in the subject 106. Example configurations of the modules 152 and 154 are illustrated in FIG. 3 and FIG. 11, respectively, and processes as may be executed by the modules 152 and 154 are provided, in example configurations, by the optimization processes of FIG. 4 and FIG. 12, respectively.

Toward that end, the stored data 114 may include various types of data, including, for example, data identifying regions of interest (ROIs) in the subject 106 representing areas for targeted stimulation and regions of avoidance (ROAs) representing areas of desired avoidance of stimulation. The stored data 114 may include constraint data, constraint parameters, constraints expressions that are utilized by the single-objective optimization module 152 and/or the multi-objective optimization module 154 for optimization, along with a discretized point model onto which the optimization is to be performed. The discretized point model, as discussed in various examples, may be a point model of each ROI (and each ROA in various examples) and each electrode in the stimulator available for targeted stimulation. The discretized point model may contain a single discretized point, a line of discretized points, a two-dimensional (2D) area of discretized points, or a volume (three-dimensional (3D)) of discretized points. The discretized point model may contain different types of discretized points for the ROI compared to the ROA. In some examples, the discretized point model is a FEM model. More generally, any suitable analytical or numerical solutions may be used to develop discretized point models herein. The stored data 114 further includes stimulation parameters generated by the single-objective optimization module 152 and/or the multi-objective optimization module 154 to be used by the stimulation controller 156 to configure and operate electrodes in the stimulator 104, in accordance with processes and methods herein.

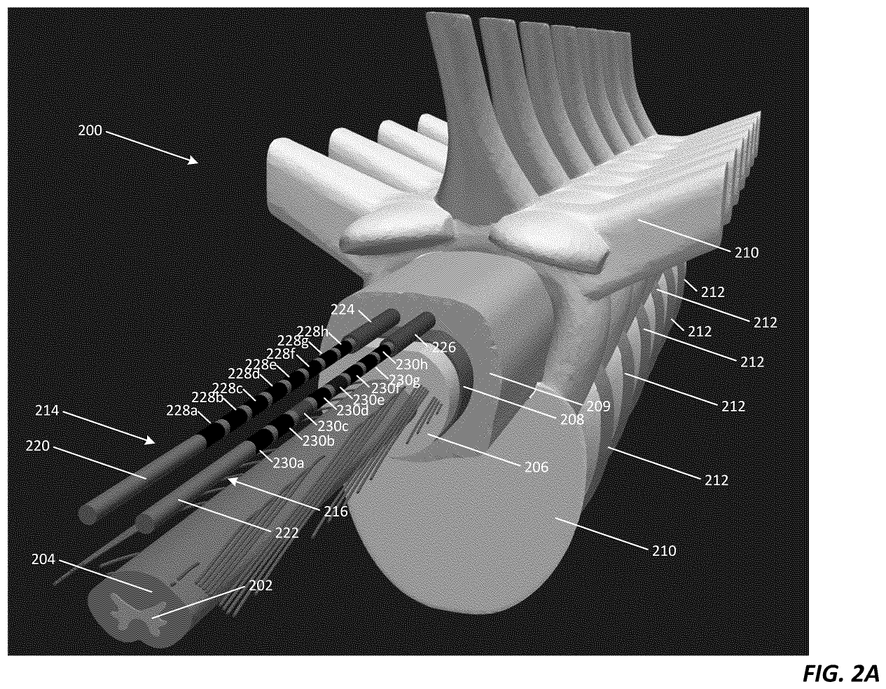

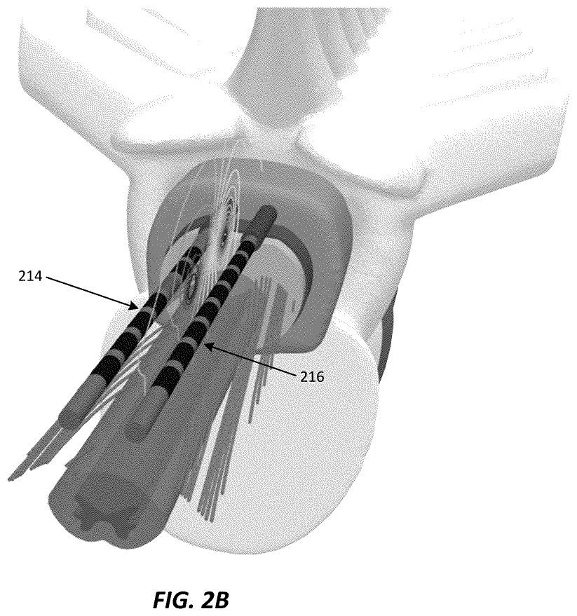

FIG. 2A illustrates a portion of a spinal cord and surrounding anatomy 200 that may be modeled using a discretized point model, in accordance with various examples. Grey matter 202 forming a hornlike structure surrounded by white matter 204 extend longitudinally surrounded by cerebrospinal fluid 206, within the dura 208 surrounded by extradural tissue 209. The spine bone structure 210 includes intervertebral discs 212.

To affect stimulation, an implanted stimulator may be formed of one or more electrode arrays for therapy. Further, the implanted stimulators may be formed of different types and different styles of electrode arrays from those shown. In the illustrated example of FIG. 2A, the implanted stimulator is formed of two electrode arrays 214 and 216, each implanted generally in parallel to one another and each extending longitudinally along a z-axis coinciding with the longitudinal axis of the dura 208. The electrode arrays 214/216 are formed of conductive wires running through a central core surrounded by insulation 220/222 (e.g., formed of an insulating material such as polyurethane or other biologically inert electrically insulating material) and a series of electrodes, e.g., electrodes 228a-228h and 230a-230h, respectively, that are each individually controllable by a stimulation controller for targeted neural stimulation of a ROI. The electrodes 228 and 230 may be formed of conductive material such as metallic material, e.g., a Platinum/Iridium (PtIr) alloy (90% Pt, 10% Ir). However, other metallic materials may be used, and, in some examples, non-nonmetallic electrodes or coatings may be used to form the electrodes 228 and 230 (e.g., polymer coating). Encapsulation layers 224/226 are also shown and may represent fibrotic scar tissue-a fibrous tissue capsule around the implant that forms as part of the body's inflammatory response to the implanted electrodes. In other examples, the layers 224/226 could also represent edema or fluid around the electrodes.

To illustrate differences in stimulation configurations, in FIG. 2A all electrodes are inactive, except electrode 230c is controlled to operate as a cathode electrode and electrode 230e as an anode electrode. In this way, FIG. 2A provides an exploded view of an example FEM model generated for applications for SCS. FIG. 2B illustrates an example resulting total electric potential field targeted stimulation that results from another stimulation configuration. That is, FIG. 2B illustrates an example solution of the FEM model showing iso-potential lines for an example stimulation configuration generated by this optimization techniques herein. In the illustrated example, the affected ROI is a single axon symmetrically positioned between two electrodes applied an optimized stimulation.

FIGS. 3 and 11 illustrate example implementations of the single-objective optimization module 152 and the multi-objective optimization module 154, respectively.

Referring to FIG. 3, in an example single-objective optimization module 300 includes a programming interface 302, such as for example, an application providing a graphical user interface (GUI) displayed to a user. That GUI interface may provide a visual display for a model of a sample region within a subject, such as an organ, tissue, anatomical structure, etc., where the visual display may be a 2D image such as a cross-sectional image or a 3D image. The image may be a generated image, such as a template image or other canonical image. The image may be an actual medical image obtained from a subject, including an annotated image thereof. In the example of spinal neural stimulation, the anatomical structure displayed may be an image of the spinal cord 200, as shown in FIG. 2A. In the example of FIG. 3, a user selects a ROI within the display using a ROI selector 304. In some examples, if a user selects multiple ROIs, then they may be treated as collectively forming a ROI for single-objective optimization. Further, in some examples, the user provides a field selector 306, which may be a gradient of the electric potential field for terminal stimulation or the second derivative of the electric potential field (activating function) for axonal stimulation. In some examples, the user may select additional field variables using the field selector 306. Each of the ROI selector 304 and the field selector 306 are shown as examples of a user input 308. These selection inputs 308 are provided to a canonical/patient-specific computational model 310 in a ROI optimizer module 312, which, as further described herein, includes a single-objective optimization algorithm 314 configured to generate stimulation parameters 316 that are stored and provided to a stimulation controller 318 for controlling the implantable stimulator 104.

FIG. 4 illustrates an example process 400 for controlling targeted neural stimulation to a subject for treatment and as may be implemented using the environment 300, including the single-objective optimization module 300. At a block 402, the process determines stimulation signals for each electrode positioned for stimulation, for example, the one or more electrodes forming electrode arrays 214/216 in FIG. 2A. The block 402 may determine stimulation signals, as signal data, by accessing a stored discretized point model representative of each ROI. In some examples, that discretized point model models each ROI as well as the plurality of electrodes available for targeting the respective ROIs. In the illustrated example, the block 402 may implement process 500 (“M0”) or process 600 (“M1”), as discussed further below.

At a block 404, the process 400 establishes an optimization problem and one or more expressions of the optimization problem. The optimization problem at block 404 may be determined using a stimulation signal superposition model of each of the electrodes relative to each discretized point in the discretized point model of the block 402. For example, the block 404 may identify cumulative field effects at each of the plurality of discretized points with a ROI. In some examples, the blocks 402 and 404 generate a matrix of different combinations of electrode stimulation signals and resulting superpositions at discretized points in a ROI. In the illustrated example, the block 404 may implement process 700 (“M2”), as discussed further below.

At a block 406, the process 400 performs an optimization routine to find the stimulation signal data that optimizes a superposition field at the ROI according to an objective function. In some examples, the block 406 generates stimulation parameters to optimize targeted stimulation to a ROI, or multiple sets of stimulation parameters when targeting multiple ROIs. In the illustrated example, the block 406 may implement process 800 (“M3”), as discussed further below.

At a block 408, a stimulation controller receives the stimulation parameters and applies targeted stimulation to a subject based on those parameters, during a therapeutic treatment operation. In some examples, the block 408 may generate a 3D model of the ROI and display a visualization of the stimulation signals or the resulting field within an ROI. In various examples, the block 408 may display the suggested optimized stimulation parameters before applying them to the subject, for review by clinicians or other personnel. Further, in some examples, the block 408 may store the optimization problems and solutions of the process 400, for access later, including for future treatment or comparison to future optimizations of the process 400.

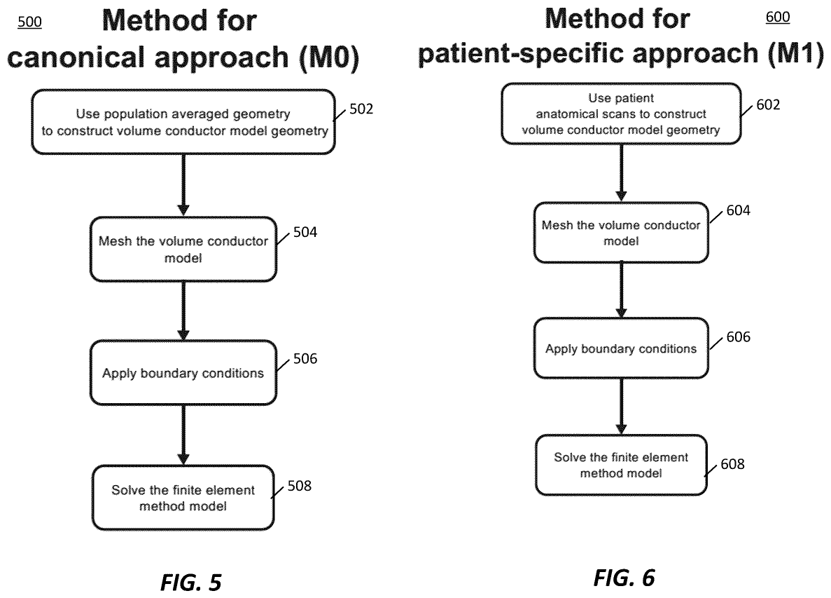

FIGS. 5 and 6 illustrate example implementations of the block 402. FIG. 5, for example, provides a canonical process 500 for obtaining stimulation signals for each electrode. At a block 502, a targeted neural stimulation platform, such as platform 150, and a single-objective optimization module 152 or 300, obtains from a stored location, average geometry data for a population and constructs a volume conductor model geometry, which is the anatomy of the canonical model process (500) including proper placement of stimulating electrodes. At a block 504, a mesh or discretization process is applied to the volume conductor model of block 502 to provide a geometric processing that converts the volume conductor model geometry to a series of geometric surfaces and volumes, e.g., polygonal surfaces and tetrahedrons. The size of the mesh elements may be predetermined and determines the level of detail and computational cost, which can be mitigated by offloading processing offline or to a cloud server. At a block 506 boundary conditions are applied to the mesh from the block 504 to bound the modeled geometry from the block 502. Finally, at a block 508, a finite element method (FEM) is applied from which a FEM model of all or some portion of the stimulation probe and surrounding regions is determined and stored. Generally, the FEM model may be solved for where the ROIs and ROAs are located or where a visualization of the resulting field is desired, therefore an FEM model need not extend to includes regions beyond these to perform optimization. In some examples, the block 508 may solve the FEM model for the entire model, and the block 508 may store only the solved FEM model for the potential ROIs and ROAs (if applicable). That is, the FEM models herein may include more than the ROI(s), the electrodes, and the ROA(s). However, the processes herein can perform optimization for the ROI(s) and ROA(s), specifically, therefore that portion of the FEM solution corresponding to the ROI(s) and ROA(s) may be determined and stored, without the solutions corresponding to the other portions of the FEM model. Further, in some examples, those optimizations may exclude the electrodes, for example, with example SCS implementations, while other optimizations may include the electrodes (for example, with example deep brain stimulation implementations).

In contrast to the process 500, FIG. 6 provides a patient-specific process 600 for obtaining stimulation signals for each electrode. At a block 602, a targeted neural stimulation platform, such as platform 150, and in particular a single-objective optimization module 152 or 300, obtains from anatomical images of a patient, such as radiography, computed tomography (CT), magnetic resonance imaging (MRI), or ultrasound and constructs a volume conductor model geometry, which is the anatomy of the patient including the stimulation electrode and may be determined by a clinician or other expert using a software interface. That is, the process 602 may be manually performed, fully automated, or a combination thereof. The relevant anatomy and electrodes may be segmented, co-registered (if necessary), and the segmentations may then be used to generate the volume conductor model. These segmentations are converted from masks to surfaces and/or volumes that are used in the volume conductor model. Other techniques may be used to construct the model geometry at block 602.

Similar to the process 500, at a block 604, a mesh or discretization process is applied to the volume conductor model of block 602 to provide a geometric processing that converts the volume conductor model geometry to a series of geometric volumes (e.g., tetrahedrons) and geometric surfaces (e.g., polygonal surfaces or equal or dissimilar numbers of edges each surface). The size of the mesh elements may be predetermined and determines the level of detail and computational cost. At a block 606, boundary conditions are applied to the mesh from the block 604 to bound the modeled geometry from the block 602. Finally, at a block 608, a FEM is applied from which a FEM model of all or some portion of the stimulation probe and surrounding regions is determined and stored. Generally, the FEM model may be solved for where the ROIs and ROAs are located or where a visualization of the resulting field is desired. In some examples, the block 608 may solve the FEM model for entire model and the block 608 may store only the solved FEM model for the potential ROIs and ROAs (if applicable).

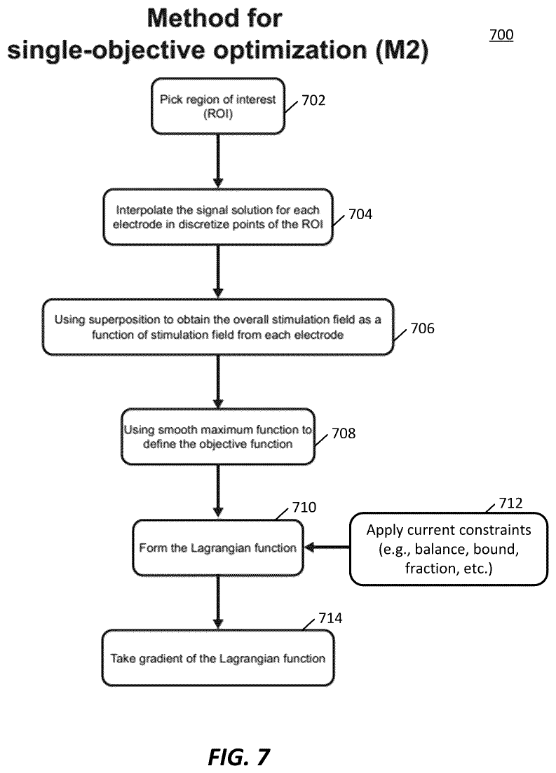

FIG. 7 illustrates an example implementation of the block 404 from FIG. 4. In particular, the process 700 determines a single-objective optimization problem for setting forth stimulation conditions to be applied at a ROI. A different single-objective optimization problem may be set forth for each ROI, if there are a series of ROIs. At a block 702, a ROI is identified, for example, by a user or through automated image feature detection and segmentation processes applied to received image data. At a block 704, the discretized point model for the ROI is obtained. Stimulation signal data from block 402 is collected, and the block 704 interpolates signal solutions for each electrode's field effect at each discretized point in the ROI. At a block 706, a superposition is performed to obtain an overall stimulation field as a function of the stimulation field from each electrode, and for each discretized point. From there, a smooth maximum function is provided at a block 708 to define an objective function that encompasses the field that needs to be maximized in the ROI as a function of electrode currents. The objective function is provided to a Lagrange transformation process to generate a Lagrange function at a block 710, where that transformation is performed by applying constraints for example a current balance constraint and current bound constraints from a block 712 to the block 710. The constraints may be current balance (among electrodes and/or between electrodes), current bound, and/or current fraction constraints, for example. In some examples, the constraints are making sure the sum of positive currents equal to +1 and the sum of negative currents equal to −1. These constraints are not directly resolvable using the Lagrange transformation process, therefore, in such examples, the constraints are converted to expressions that include some approximation to allow for use with the process described. In an example, converting these constraints to what can be used in Lagrange transformation process leads to a constraint that is current balance. Prior to conversion, the constraints amount to a bound condition, that because it has absolute value, may be approximated. Continuing, once the Lagrange function is formed, at a block 714, the gradient of the Lagrange function is determined.

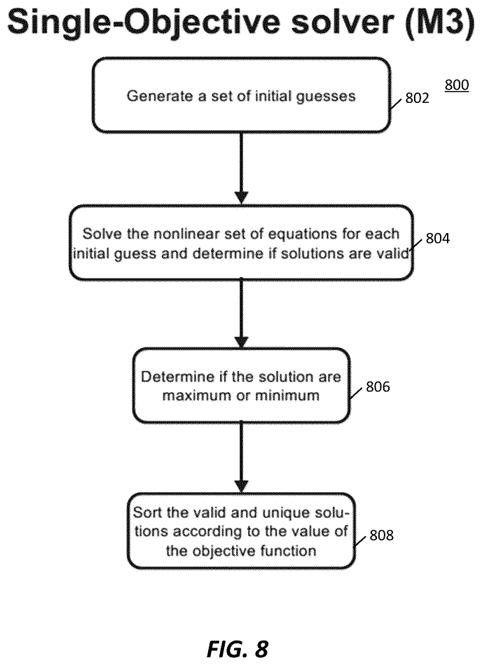

FIG. 8 illustrates an example implementation of the block 406 from FIG. 4, showing a process for solving the optimization problem defined at block 404. A process 800 begins at a block 802 that generates an initial set of guesses for a solution to the optimization problem of block 404, which are defined as a set of a nonlinear equations. For example, these guesses may be a set of stimulation conditions, defining parameters, i.e., the current fractions applied at the individual electrodes. A block 804 attempts to solve the set of nonlinear equations for each initial guess in the set and determines if the solutions are valid. At a block 806, the resulting solution being a maximum, a minimum, or none is determined, and the block 808 is configured to identify either a maximum solution or a minimum solution as the optimal solution among a set of solutions depending on the optimization problem defined at the block 404. The valid and unique solutions are then sorted according to the resulting value of the objective function, where that sorting may be in a ranking order from highest maximum or from lowest minimum, for example. Maximums and minimums can be local or global. Block 806 may determine if a solution is a maximum or a minimum mathematically. Some solutions are neither maximum nor minimum but rather are “none” (e.g., saddle points), and the block 806 may discard those. In block 808, these solutions may be sorted to rank them and identify which is a global maximum/minimum. The solutions can be equally good (equal or close objective functions). In some examples, at the block 808, the user can decide to pick one based on criteria of their choosing or try them all and see if one is better than other based on something they observe in real time (e.g., a patient has some preference, or one solution provides better outcome compared to the other).

### Example Single-Objective Optimization

An example implementation of the single-objective optimization module 300 and/or the process 400 is now described. In this example, the single-objective optimization problem included maximizing the maximum of the field ( [symbol] ) that leads to the activation of axons in the ROI:

$$\begin{matrix}maximize : max ( ℱ ( X ) ) & X ∈\end{matrix} ROI , \qquad ( 1 )$$

where X represents the spatial points in the ROI. The field ( [symbol] ) can be estimated as the first derivative of the electric potential ( [symbol] =−dV/dr) for terminal excitation or the second derivative of the electric potential ( [symbol] =d²V/dr²) for axonal excitation where (r) is the direction along the target structure.

To solve the optimization problem, process 400 is configured to evaluate F as a function of the current at each electrode. Assuming quasi-static conditions, the superposition principle can be used to evaluate [symbol] :

$$ℱ ( X , α ) = {\sum }_{i = 1}^{n} {α}_{i} {i}_{} ( X ) , \qquad ( 2 )$$

where αᵢ is the current at the i^{th} contact, n is the total number of contacts, and [symbol] (X) is the field obtained from the solution of the FEM model when the current at the i^{th} contact is equal to a unit current of 1 A while the current at all other contacts are 0 A.

Without any constraints, the solution of Eq. 1 is not bounded because increasing α leads to the increase in [symbol] . Therefore, α can be considered to represent current fractions rather than the actual currents (i.e., values of α varies between −1 and +1). It can also be assumed that the total inward (α<0) and outward (α>0) current fractions are balanced:

$${\sum }_{i}^{n} {α}_{i} = + \begin{matrix}1 & if\end{matrix} {α}_{i} > 0. \qquad ( 3 )$$

$${\sum }_{i}^{n} {α}_{i} = - \begin{matrix}1 & if\end{matrix} {α}_{i} < 0. \qquad ( 4 )$$

Therefore, the single-objective optimization problem in this example includes solving Eq. 1 along with Eqs. 3 and 4 as constraints. Solving this optimization problem can be challenging because the maximum operator in Eq. 1 is nondifferentiable, which prevents using analytical optimization methods, as do the conditional statements in Eqs. 3 and 4. To overcome this issue for the objective function, the maximum operator can be replaced with a smooth function that approximates the maximum of the field:

$${max}_{S} ( ℱ ( X , α ) , β ) = \frac{{\sum }_{i = 1}^{m} ℱ ( {X}_{i} , α ) exp ( βℱ ( {X}_{i} , α ) )}{{\sum }_{i = 1}^{m} exp ( βℱ ( {X}_{i} , α ) )} , \qquad ( 5 )$$

where m is the number of spatial points in the ROI, and β>0 is a scaling parameter. For sufficiently large β, Eq. 5 can approximate the maximum of the field in the ROI. In practice, β does not need to go to infinity, and as long β [symbol] (Xⱼ,α) is sufficiently large, then Eq. 5 can provide a smooth approximation to the maximum operator. Moreover, choosing β<0 allows for finding the largest negative value of the Eq. 5. In fact, Eq. 5 produces the exact maximum for β->infinity.

To resolve the issue with the conditional statements for the constraints (Eqs. 3 and 4), Eq. 3 and Eq. 4 can be summed:

$${\sum }_{i}^{n} {α}_{i} = 0. \qquad ( 6 )$$

Equation 6 plainly represents the constraint that the currents need to be balanced. However, the information that the current fractions are between +1 and −1 is lost when Eqs. 3 and 4 are summed together. However, the absolute values of Eqs. 3 and 4 can be calculated and then summed together:

$${\sum }_{i}^{n} \vert {α}_{i} \vert = 2. \qquad ( 7 )$$

Equation 6 is well-posed and allows the use analytic methods. However, Eq. 7 is problematic because it is not differentiable at 0, but the absolute value function can be replaced with a smooth approximation:

$${abs}_{S} ( α , γ ) = αtanh ( γα ) , \qquad ( 8 )$$

where γ>0 is a scaling parameter. Hence, Eq. 7 can be approximated as:

$${\sum }_{i}^{n} {α}_{i} tanh ( {γα}_{i} ) = 2. \qquad ( 9 )$$

Replacing the maximum operator and the absolute value function with their smooth approximation (Eq. 5 and Eq. 9, respectively) allows the Lagrange multipliers method to be used to solve the optimization problem. To that end, the single-objective optimization problem can be formulated as:

$$\begin{matrix}maximize : {max}_{S} ( ℱ ( X , α ) , β ) & X ∈\end{matrix} ROI$$

$$subject to : {h}_{1} ( α ) = 2 - {\sum }_{i = 1}^{n} {α}_{i} tanh ( {γα}_{i} ) = 0 and {h}_{2} ( α ) = {\sum }_{i = 1}^{n} {α}_{i} = 0 , \qquad ( 10 )$$

where [symbol] ₁ and [symbol] ₂ represent the equality constraints. Next, the Lagrangian function can be written as:

$$ℒ ( α , λ ) = {max}_{S} ( ℱ ( X , α ) , β ) - {\sum }_{i = 1}^{2} {λ}_{i} {h}_{i} ( α ) , \qquad ( 11 )$$

where λ is the Lagrange multiplier for the equality constraints. The critical points of the Lagrangian function can be found by taking its gradient and setting it equal to 0:

$${∇}_{α , λ} ℒ ( α , λ ) = 0. \qquad ( 12 )$$

Equation 12 yields a system of n+2 equations (n current fractions and 2 equality constraints). This system of equations is solved to find the critical points of the Lagrangian function. Finally, the sequence of minors of the bordered Hessian matrix is used to determine if these critical points are maximums, minimums, or none (saddle points), as may be performed by block 806.

The foregoing example describes operations that may be performed by process 400, including that of the process 800. Referring to FIG. 8, in some examples, the block 808 may perform post solution data processing, such as rounding the values of the solution and/or performing minor corrections. For example, current fractions that are obtained from block 712 or otherwise generated can be fractional numbers, such as 0.9995 and −0.996 for example. The block 808 may perform rounding and may also ensure that the solutions satisfy the true constraints (see, e.g., Eq. 3 and 4)). The current fractions may not satisfy the constraint because of rounding or/and because of the approximation in Eq. 9 plus some numerical inaccuracies. Therefore, the block 808 may be configured to correct current values until such constraints are satisfied.

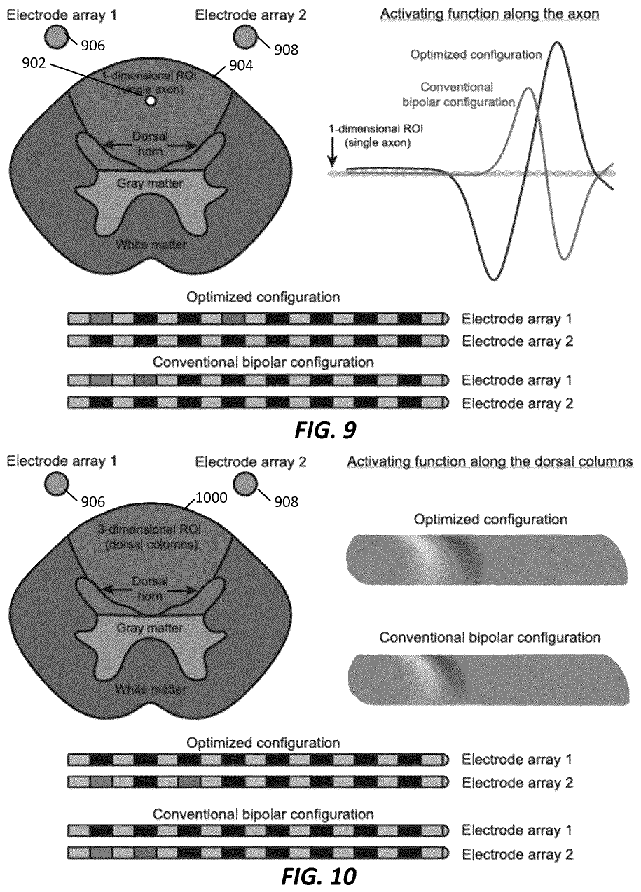

FIG. 9 is an illustration of an example ROI and resulting stimulation configuration, applying a single-objective optimization. In the illustrated example, the ROI is a one-dimensional (1D) ROI, in particular an individual axon 902 within the dorsal columns 904 of the spinal cord. In this example, the single-objective optimization seeks to maximize the activating function (i.e., second-order spatial derivative of the applied electric potential field) along the axon by determining a stimulation configuration to be applied by the electrode arrays 906 and 908. In this example, along the axon refers to the direction that the second derivative of the applied electric potential field is calculated along (taking the spatial derivative in that direction twice). FIG. 9 shows the activating function (i.e., second-order spatial derivative) of the applied electric potential field along the axon for the optimized configuration and for a standard bipolar configuration.

In the illustrated example of FIG. 10, the single-objective optimization was applied to a 3D ROI (i.e., the volume corresponding to the dorsal columns), where the optimization is to maximize the activating function within the entire 3D ROI volume 1000, using the electrode arrays 906 and 908. That is, FIG. 10 is similar to FIG. 9 except FIG. 10 shows the activating function throughout the dorsal columns (in the rostral-caudal direction) as opposed to a single axon as shown in FIG. 9.

FIG. 11 illustrates an example multi-objective optimization module 1300 that may be used as the multi-objective optimization module 154 in the environment 100 of FIG. 1. For example, the optimization module 1300 may be used to provide control signals to a plurality of electrodes and based on stimulation signal parameters affect treatment of the region of interest and of the region of avoidance by those electrodes, in accordance with example processes herein. The multi-objective optimization 1300 that includes a programming interface 1302, such as for example, an application providing a graphical user interface (GUI) displayed to a user. That GUI interface may provide a visual display for a model of a sample region within a subject, such as an organ, tissue, anatomical structure, etc., where the visual display may be a 2D image such as a cross-sectional image or a 3D image. The image may be a generated image, such as a template image or other canonical image. The image may be an actual medical image obtained from a subject, including an annotated image thereof. In the example of spinal neural stimulation, the anatomical structure displayed to a used may be an image of the spinal cord 200, as shown in FIG. 2A.

In FIG. 11, a user selects one or more ROIs within the display using a ROI selector 1304. Further, the user provides a field selector 1306, e.g., gradient of the electric potential field for terminal stimulation or second derivative of the electric potential field (activating function) for axonal stimulation. Similar to the single-objective optimization module 300, each of the ROI selector 1304 and the field selector 1306 are shown as examples of a user input 1308. The multi-objective optimization module 1300 further includes other objective conditions, beyond the ROI(s) for optimized stimulation. In the illustrated example, these other objective conditions are indicated as ROAs 1309 and corresponding ROAs field selections 1307, that are identified by the user. Each of these are additionally included in the collective selection input 1308. This selection input 1308 is provided to a canonical/patient-specific computational model 1310 in a ROI/ROA optimizer module 1312, which, as further described herein, includes a multi-objective optimization algorithm 1314 configured to generate a Pareto front of stimulation parameters 1316 that are stored and provided to a stimulation controller 1318 for controlling the implantable stimulator 104. Indeed, references herein to ROI in the context of a multi-objective optimization may be considered as including both ROI(s) and ROA(s), e.g., electric potential fields, selections, models, etc.

FIG. 12 illustrates an example process 1400 for controlling targeted neural stimulation to the subject for treatment and as may be implemented using the environment 1300, in particular, including the multi-objective optimization module 1300. At a block 1402, the process determines stimulation signals for each electrode positioned for stimulation, for example, the one or more electrodes forming electrode arrays 214/216 in FIG. 2A. The block 1402 may determine stimulation signals, as signal data, by accessing a stored discretized point model representative of each ROI. In some examples, that discretized point model models each ROI. The block 1402 is some examples may be implemented by processes 500 (“M0”) or 600 (“M1”), discussed herein.

At a block 1404, the process 1400 establishes an optimization problem, for example, in the form of an expression formed of one or more equations. The optimization problem at block 1404 may be determined using a stimulation signal superposition model of each of the electrodes relative to each discretized point in the discretized point model of the block 1402. For example, the block 1404 may identify cumulative field effects at each of the plurality of discretized points within a ROI as well within a ROA. In some examples, the blocks 1402 and 1404 generate a matrix of different combinations of electrode stimulation signals and resulting superpositions at discretized points in each ROI and at discretized points in each ROA. In the illustrated example, the block 1404 may implement process 1500 (“M4”), as discussed further below.

At a block 1406, the process 1400 performs an optimization on the stimulation signal data to optimize a superposition field at each ROI while minimizing the superposition field at each ROA, according to an objective function. In some examples, the block 1406 generates stimulation parameters to optimize targeted stimulation to a ROI, or multiple sets of stimulation parameters when targeting multiple ROIs. At a block 1408, a stimulation controller receives the stimulation parameters and applies targeted stimulation to a subject based on those parameters. In the illustrated example, the block 1406 may implement process 1600 (“M5”), as discussed further below.

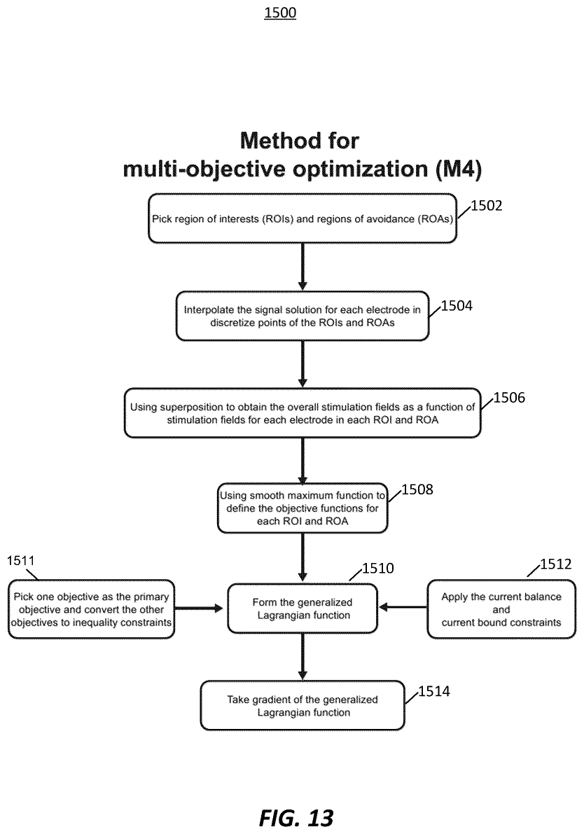

FIG. 13 illustrates an example implementation of the block 1404 from FIG. 12. In particular, the process 1500 determines a multi-objective optimization problem for setting forth stimulation conditions to be applied at ROIs and ROAs. At a block 1502, one or more ROIs and ROAs are identified, for example, by a user or through automated image feature detection and segmentation processes applied to received image data. At a block 1504, the discretized point model for the ROI(s) and the ROA(s) is obtained. Stimulation signal data from block 1402 is collected, and the block 1504 interpolates signal solutions for each electrode's field effect at each discretized point in the ROI(s) and at each discretized point in the ROA(s). At a block 1506, a superposition is performed to obtain an overall stimulation field as a function of the stimulation field from each electrode, and for each discretized point. From there, a smooth maximum function is provided at a block 1508 to define an objective function for each ROI and for each ROA, where the objective functions encompass the field that needs to be maximized in the ROI as a function of electrode currents. The objective function is provided to a Lagrange transformation process to generate a Lagrange function at a block 1510, where in some examples, that transformation is performed by applying a current balance constraint and current bound constraints from a block 1512 to the block 1510 and by determining an objective function as the primary objective function and converting the other objective functions to inequality constraints at a block 1511. As with the single-objective optimization, the constraints may be current balance (among electrodes and/or between electrodes), current bound, and/or current fraction constraints, for example. In some examples, the constraints are making sure the sum of positive currents equal to +1 and the sum of negative currents equal to −1. These constraints are not directly resolvable using the Lagrange transformation process, therefore, in such examples, the constraints are converted to expressions that include some approximation to allow for use with the process described. In an example, converting these constraints to what can be used in the Lagrange transformation process leads to a constraint that is current balance. Prior to conversion, the constraints amount to a bound condition, that because it has absolute value, may be approximated. Once the Lagrange function is formed, at a block 1514 the gradient of the Lagrange function is determined.

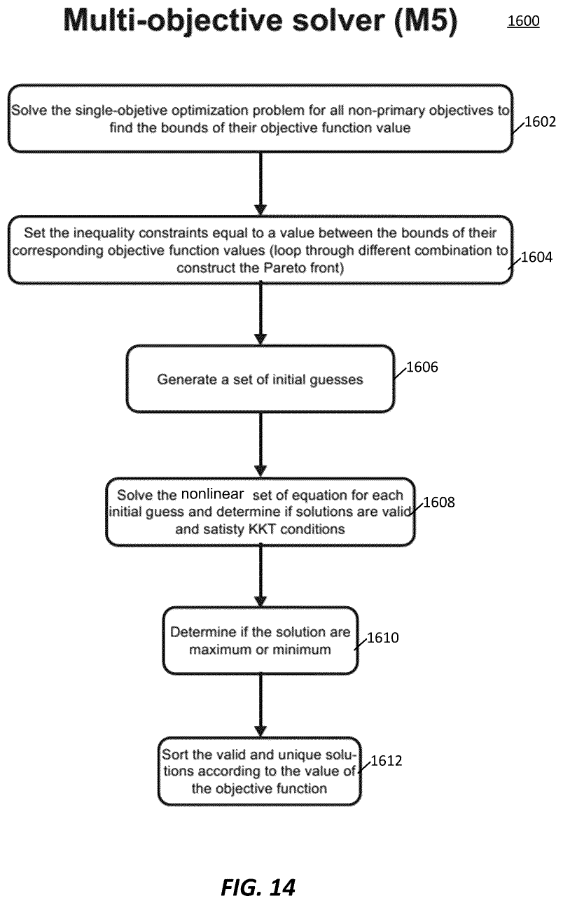

FIG. 14 illustrates an example implementation of the block 1406 from FIG. 12, showing a process for solving the optimization problem defined at block 1404. At a block 1602, the process 1600 solves a single-objective optimization problem, for example, a primary objective function identified at block 1511 in FIG. 13. In the illustrated example, the process 1602 solves that single-objective optimization problem for all non-primary objectives to find the bounds of the objective function value. At a block 1604, inequality constraints are determined and set to a value between the bounds of their corresponding objective function values (e.g., from the block 1510 in FIG. 13). For example, the process 1604 may be configured to identify inequality constraints by looping through different combinations to construct a Pareto front.

At a block 1606, the process 1600 generates an initial set of guesses for a solution to the optimization problem of block 1602. A block 1608, the process 1600 attempts to solve the set of nonlinear equations for each initial guess in the set and determines if the solutions are valid, and, in the illustrated example, satisfy Karush-Kuhn-Tucker (KKT) conditions (see, e.g., Equations (16)-(20)). At the block 1610 the resulting solution being a maximum, a minimum, or none is determined, and the block 1612 is configured to identify either a maximum solution or a minimum solution as the optimal solution among a set of solutions depending on the optimization problem from the bock 1602. The maximums and minimums can be local or global. At the block 1612, the valid and unique solutions are then sorted according to the resulting value of the objective function, where that sorting may be in a ranking order from highest maximum or from lowest minimum, for example. In block 1612, these solutions may be sorted to rank them and identify which is a global maximum/minimum. The solutions can be equally good (equal or close objective functions). In some examples, at the block 1612, the user can decide to pick one based on criteria of their choosing or try them all and see if one is better than other based on something they observe in real time (e.g., a patient has some preference, or one solution provides better outcome compared to the other).

### Example Multi-Objective Optimization

An example implementation of the multi-objective optimization module 1300 and/or the process 1400 is now described. In an example, the single-optimization framework of FIG. 3 is extended to solve multi-objective optimization problems for more complex target stimulation configurations.

In this example, two types of multi-objective optimizations are considered: a) maximizing the fields that leads to excitation in more than one ROI, and b) maximizing the fields that leads to excitation in the ROIs and minimizing the fields that leads to excitation in regions of avoidance (ROAs). Note that the field that leads to activation can be different in each ROI and ROA (e.g., first or second derivatives of the electric potential in different directions).

In the case of maximizing different fields in ROIs (“case a”), the objective function in each ROI is similar to Eq. 1. However, the objective for minimizing the excitation fields in ROAs is:

$$\begin{matrix}maximize : max ( ℱ ( X ) ) & X ∈\end{matrix} ROA . \qquad ( 13 )$$

A method of solving multi-objective optimization problems is to combine them into a single objective as a weighted sum. Then, the tradeoff between different objectives can be explored by varying the weight of each objective. However, this approach is prone to failure for multiple reasons (e.g., if the Pareto front is nonconvex or the objectives are not properly scaled). Therefore, in this example multi-optimization module, the module was configured with another approach, using an epsilon, ∈-constraint method. In this approach, a single objective is considered as the primary objective and all the other objectives are considered as inequality constraints:

$$\begin{matrix}maximize : {max}_{S} ( {ℱ}_{1} ( X , α ) , β ) & X ∈\end{matrix} ROI$$

$$\begin{matrix}subject to : {g}_{i} = {max}_{S} ( {ℱ}_{1} ( X , α ) , β ) - {ϵ}_{i} \leq 0 & X ∈\end{matrix} {ROI}_{i} or {ROA}_{i} \qquad ( 14 )$$

where i≥2 is the index of the i^{th} ROI or ROA. Then, E can be varied to construct the Pareto fronts even if they are not convex.

The generalized Lagrange multipliers method is used to solve the new single-objective optimization problem, which includes inequality and equality constraints (Eqs. 6 and 9). For a given ∈, the Lagrangian expression can be written as:

$$ℒ ( α , λ , μ ) = {max}_{S} ( {ℱ}_{1} ( X , α ) , β ) - {\sum }_{i = 1}^{2} {λ}_{i} {h}_{i} ( α ) - {\sum }_{i = 2}^{Q} {μ}_{i} {g}_{i} ( α , {ϵ}_{q} ) , \qquad ( 15 )$$

where λ and μ are the Karush-Kuhn-Tucker (KKT) multipliers, and Q is the total number of ROIs or ROAs. The critical points of the generalized Lagrangian function can be found by using the KKT conditions:

$${∇}_{α , λ , μ} ℒ ( α , λ , μ ) = 0 \qquad ( 16 )$$

$$g ( {α}^{*} , ϵ ) \leq 0 \qquad ( 17 )$$

$$h ( {α}^{*} ) \leq 0 \qquad ( 18 )$$

$${μ}^{*} \geq 0 \qquad ( 19 )$$

$${\sum }_{i = 2}^{Q} {μ}_{i}^{*} {g}_{i} ( {α}^{*} , {ϵ}_{q} ) = 0 \qquad ( 20 )$$

where α* and μ* are a solution of the system of equations obtained from Eq. 16. The solution set is valid only if it satisfies conditions shown in Eqs. 17-20. Next, the sequence of minors of the bordered Hessian matrix is used to determine if the valid critical points are maximums, minimums, or none (saddle points).

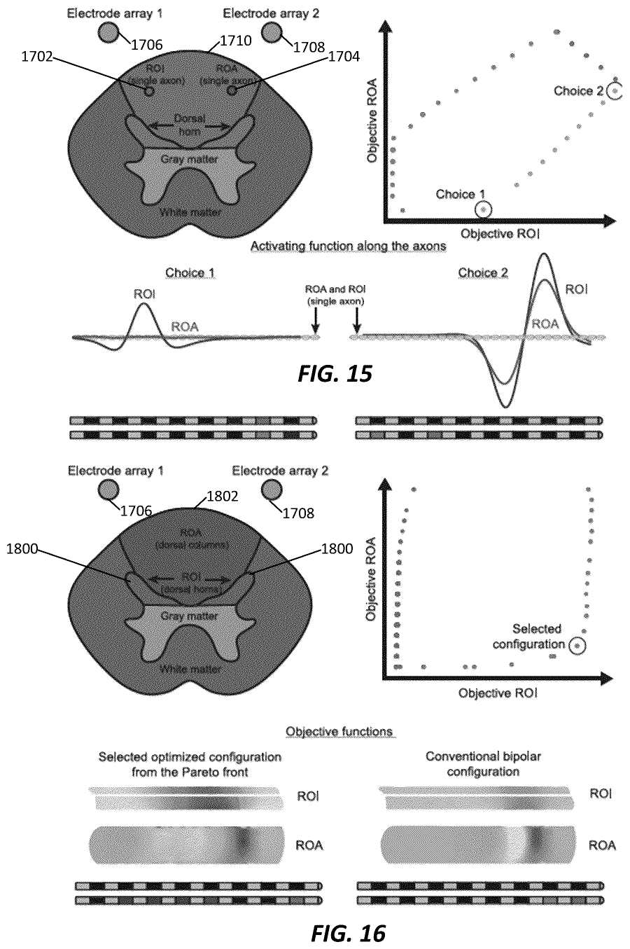

FIG. 15 is an illustration of an example ROI and resulting stimulation configuration, applying a multi-objective optimization. In the illustrated example, the ROI is a one-dimensional (1D) ROI, in particular an individual axon 1702, and the ROA is a 1D ROA, another axon 1704, within the dorsal columns 1710 of the spinal cord. In this example, the multi-objective optimization seeks to maximize the activating function (i.e., second-order spatial derivative of the applied electric potential field) along the axon 1702 while minimizing activation along the axon 1704 by determining a stimulation configuration to be applied by the electrode arrays 1706 and 1708. To determine the optimum stimulation configurations two different solution choices are provided (“Choice 1” and “Choice 2”). Choice 1 is an example of a point on the Pareto front where the objective function in the ROA is close to zero but the objective function in the ROI is nonzero but small. Choice 2 is an example of a point on the Pareto front that shows to have a greater objective function in the ROI, but the objective function in the ROA might also increase. This shows the importance of multi-objective optimization and providing the Pareto front to the user to control the tradeoff between activating the ROI and activating ROA. The activating functions along each of the axons (the ROI axon 1702 and the ROA axon 1704) are plotted as shown.

In the illustrated example of FIG. 16, the multi-objective optimization was applied to a 3D ROI corresponding to the right and left dorsal horn 1800, where the optimization is to maximize the activating function within the entire 3D ROI volume 1800, while minimizing activation in a 3D ROA 1802 corresponding to the dorsal columns, using the electrode arrays 1706 and 1708.

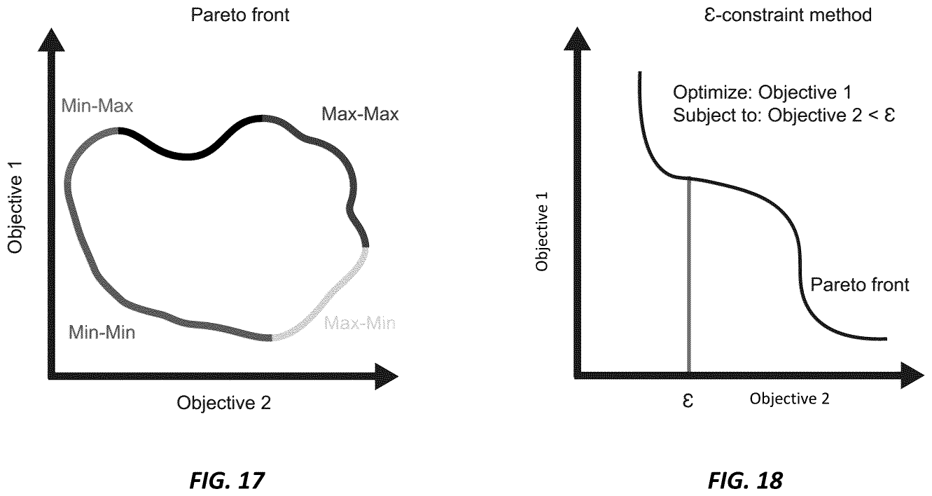

As mentioned, the multi-objective optimization modules may identify a Pareto front of different possible solutions and their effects on different objective functions. FIG. 17 illustrates a Pareto front plot for two different objectives, Objective 1 and Objective 2, and all the four different combinations of solutions for the two objectives (Objective 1| Objective 2): MIN| MAX, MIN| MIN, MAX| MAX, and MAX| MIN. Plotting the solutions resulting during the multi-objective optimization modules results in the example Pareto front, which shows tradeoffs between trying to optimize each of the two objective functions. In an example, the multi-objective optimization modules use an epsilon (ε)-constraint method to create the Pareto fronts (see, e.g., FIG. 18). In this method, one of the objectives is picked as the primary objective (e.g., Objective 1 in FIG. 18). All the other objectives (e.g., Objective 2 in FIG. 18) are considered as inequality constraints. The multi-objective optimization modules vary epsilon, ε, to construct the fronts. In FIG. 18, the range of epsilon, ε, was picked by solving the optimization problem for Objective 2 and finding the MAX and MIN of that objective function. In various examples of the multi-objective optimization, there are inequality constraints. Therefore, in some examples, the techniques use the generalized Lagrange Multiplier method with KKT conditions.

Thus, the present application describes an optimization framework to find stimulation configurations that can target specific neural structures, for example, for axonal and/or terminal stimulation. The optimization framework is able to achieve the lowest activation thresholds in ROIs. Where activation thresholds in the ROIs are not possible without activating structures within ROAs, the optimization framework may include modules that generate stimulation configurations that allow for activation within the ROIs at higher thresholds with only small levels of activation in the ROAs.

Further, the techniques herein can be used in various applications. These include systems and methods to determine where to implant the electrodes (e.g., surgical planning-run simulations to determine the placement that would maximize the desired effects in the ROIs). Others include systems and methods to determine the current amplitudes, relative strengths, polarities, etc. to apply through each electrode for given positions (e.g., for use after the electrodes have already been implanted). Indeed, yet others include patient-specific modeling into systems and methods for optimization of treatment.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the target matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed:

1. A method for controlling targeted neural stimulation to a subject for treatment, the method comprising:
determining, at one or more processors, a stimulation signal for each of a plurality of electrodes positioned for treatment of a region of interest, using a discretized point model representative of at least the region of interest;
determining, at the one or more processors, a stimulation superposition signal using the stimulation signals for each electrode, the stimulation superposition signal being determined for each discretized point in the discretized point model and from constraining all current fractions of the plurality of electrodes according to a maximum bound and/or a minimum bound;
from the determined stimulation signal superposition signal, performing, at the one or more processors, an optimization on the stimulation signal data to maximize a superposition field at the region of interest according to an objective function, wherein performing the optimization includes,
applying a smooth maximum operator to the objective function to form a differentiable objective function, and
applying a Lagrange multiplier to obtain the set of expressions that maximizes the objective function and satisfies the constraints on the current fractions, and
obtaining from the set of expressions, discrete points of the Lagrangian function corresponding to a maximum, a minimum, or a none value; and
identifying, from the optimization, a stimulation signal combination for controlling each of the plurality of electrodes for treatment of the subject and corresponding to the optimization of the superposition field according to the objective function; and
providing control signals to the plurality of electrodes and based on the stimulation signal parameters to affect treatment of the region of interest by the plurality of electrodes.

2. The method of claim 1, wherein the stimulation signal for each electrode is obtained using numerical methods or analytic methods.

3. The method of claim 1, wherein the discretized point model representative of the region of interest contains a single discretized point.

4. The method of claim 1, wherein the discretized point model representative of the region of interest contains a line of discretized points.

5. The method of claim 1, wherein the discretized point model representative of the region of interest contains a 2D area of discretized points.

6. The method of claim 1, wherein the discretized point model representative of the region of interest contains a volume of discretized points.

7. The method of claim 1, wherein the discretized point model is a finite element method model.

8. The method of claim 1, wherein the current fractions of all electrodes are simultaneously constrained to the maximum bound and the minimum bound during determination of stimulation superposition signal.

9. The method of claim 1, wherein the sum of all current fractions is constrained to be zero.

10. The method of claim 1, wherein the objective function is to maximize the superposition field that affects the region of interest.

11. The method of claim 10, wherein the objective function is a superposition of an electric potential field at the region of interest, a first order spatial derivative of the electric potential field at the region of interest, a second order spatial derivative of the electric potential field at the region of interest, or a directional field or vector fields maximized or minimized in principal directions or along trajectories in the region of interest.

12. The method of claim 1, wherein the objective function is to minimize the superposition field that affects the region of interest.

13. The method of claim 1, further comprising finding numerical solutions to the set of expressions to determine the discrete points of the Lagrangian function corresponding to the maximum, the minimum, or the none value.

14. The method of claim 12, wherein a bordered Hessian matrix is determined and minors of the bordered Hessian matrix are used to determine if the determined discrete points are the maximum, the minimum, or the none value.

15. A computing system for controlling targeted neural stimulation parameters for treatment of a subject, the system comprising:
one or more processors; and
one or more memories having stored thereon computer-executable instructions that, when executed, cause the computing system to:
determine, at one or more processors, a stimulation signal for each of a plurality of electrodes positioned for treatment of a region of interest, using a discretized point model representative of at least the region of interest;
determine, at the one or more processors, a stimulation superposition signal using the stimulation signals for each electrode, the stimulation superposition signal being determined for each discretized point in the discretized point model and from constraining all current fractions of the plurality of electrodes according to a maximum bound and/or a minimum bound;
from the determined stimulation signal superposition signal, perform, at the one or more processors, an optimization on the stimulation signal data to maximize a superposition field at the region of interest according to an objective function, wherein performing the optimization includes instructions that, when executed, cause the computing system to,
apply a smooth maximum operator to the objective function to form a differentiable objective function, and
apply a Lagrange multiplier to obtain the set of expressions that maximizes the objective function and satisfies the constraints on the current fractions, and
obtain from the set of expressions, discrete points of the Lagrangian function corresponding to a maximum, a minimum, or a none value; and
identify, from the optimization, a stimulation signal combination for controlling each of the plurality of electrodes for treatment of the subject and corresponding to the optimization of the superposition field according to the objective function; and
provide control signals to the plurality of electrodes and based on the stimulation signal parameters to affect treatment of the region of interest by the plurality of electrodes.

16. The computing system of claim 15, wherein the stimulation signal for each electrode is obtained using numerical methods or analytic methods.

17. The computing system of claim 15, wherein the discretized point model representative of the region of interest contains a single discretized point.

18. The computing system of claim 15, wherein the discretized point model representative of the region of interest contains a line of discretized points.

19. The computing system of claim 15, wherein the discretized point model representative of the region of interest contains a 2D area of discretized points.

20. The computing system of claim 15, wherein the discretized point model representative of the region of interest contains a volume of discretized points.

21. The computing system of claim 15, wherein the discretized point model is a finite element method model.

22. The computing system of claim 15, wherein the current fractions of all electrodes are simultaneously constrained to the maximum bound and the minimum bound during determination of stimulation superposition signal.

23. The computing system of claim 15, wherein the sum of all current fractions is constrained to be zero.

24. The computing system of claim 15, wherein the objective function is to maximize superposition field that affects the region of interest.

25. The computing system of claim 15, wherein the objective function is a superposition of an electric potential field at the region of interest, a first order spatial derivative of the electric potential field at the region of interest, a second order spatial derivative of the electric potential field at the region of interest, or a directional field or vector fields maximized or minimized in principal directions or along trajectories in the region of interest.

26. A non-transitory computer-readable storage medium storing executable instructions that, when executed by a processor, cause a computer to:
determine, at one or more processors, a stimulation signal for each of a plurality of electrodes positioned for treatment of a region of interest, using a discretized point model representative of at least the region of interest;
determine, at the one or more processors, a stimulation superposition signal using the stimulation signals for each electrode, the stimulation superposition signal being determined for each discretized point in the discretized point model and from constraining all current fractions of the plurality of electrodes according to a maximum bound and/or a minimum bound;
from the determined stimulation signal superposition signal, perform, at the one or more processors, an optimization on the stimulation signal data to maximize a superposition field at the region of interest according to an objective function, wherein performing the optimization includes instructions that, when executed, cause the computing system to,
apply a smooth maximum operator to the objective function to form a differentiable objective function, and
apply a Lagrange multiplier to obtain the set of expressions that maximizes the objective function and satisfies the constraints on the current fractions, and
obtain from the set of expressions, discrete points of the Lagrangian function corresponding to a maximum, a minimum, or a none value; and
identify, from the optimization, a stimulation signal combination for controlling each of the plurality of electrodes for treatment of the subject and corresponding to the optimization of the superposition field according to the objective function; and
provide control signals to the plurality of electrodes and based on the stimulation signal parameters to affect treatment of the region of interest by the plurality of electrodes.

27. A method for controlling targeted neural stimulation to a subject for treatment, the method comprising:
determining, at one or more processors, a stimulation signal for each of a plurality of electrodes positioned for affecting a region of interest and for affecting a region of avoidance, using a discretized point model representative of the region of interest and of the region of avoidance;
determining, at the one or more processors, a stimulation superposition signal using the stimulation signals for each electrode, the stimulation superposition signal being determined for each discretized point in the region of interest and in the region of avoidance and constraining all current fractions of the plurality of electrodes according to a maximum bound and/or a minimum bound;
from the determined stimulation signal superposition, performing, at the one or more processors, a multi-objective optimization on the stimulation signal data to maximize a superposition field at the region of interest and to minimize a superposition field at the region of avoidance according to a multi-objective function, wherein performing the optimization includes,
applying a smooth maximum operator to the objective function to form a differentiable objective function, and
applying a Lagrange multiplier to obtain the set of expressions that maximizes the objective function and satisfies the constraints on the current fractions, and
obtaining from the set of expressions, discrete points of the Lagrangian function corresponding to a maximum, a minimum, or a none value,
using an epsilon-constraint method to construct a Pareto front of solutions for the multi-objective function,
selecting an objective function of the multi-objective function as a primary objective and converting the other objective functions to inequality constraints,
applying a generalized Lagrange multiplier to obtain the set of expressions that satisfies the multi-objective function and satisfies the constraints on the current fractions; and
identifying, from the optimization, a stimulation signal combination for controlling each of the plurality of electrodes for treatment of the subject and corresponding to the optimization of the superposition field according to the objective function; and
providing control signals to the plurality of electrodes and based on the stimulation signal parameters to affect treatment of the region of interest and of the region of avoidance by the plurality of electrodes.

28. The method of claim 27, wherein the stimulation signal for each electrode is obtained using numerical methods or analytic methods.

29. The method of claim 27, wherein the discretized point model contains a single discretized point or a line of discretized points.

30. The method of claim 27, wherein the discretized point model contains different types of discretized points for the region of interest compared to the region of avoidance, wherein the different types of discretized points are selected from the group consisting of a single discretized point, a line of discretized points, a 2D area of discretized points, and volume of discretized points.

31. The method of claim 27, wherein the discretized point model contains a 2D area of discretized points.

32. The method of claim 27, wherein the discretized point model contains a volume of discretized points.

33. The method of claim 27, wherein the discretized point model is a finite element method model.

34. The method of claim 27, wherein the current fractions of all electrodes are simultaneously constrained to the maximum bound and the minimum bound during determination of stimulation superposition signal.

35. The method of claim 27, wherein the sum of all current fractions is constrained to be zero.

36. The method of claim 27, wherein the multi-objective function is to maximize the superposition field in the region of interest and to minimize the superposition field in the region of avoidance.

37. The method of claim 27, wherein the objective function is a superposition of an electric potential field at the region of interest, a first order spatial derivative of the electric potential field at the region of interest, a second order spatial derivative of the electric potential field at the region of interest, or a directional field or vector fields maximized or minimized in principal directions or along trajectories in the region of interest.

38. The method of claim 27, wherein the multi-objective function is to maximize the superposition field in the region of interest and maximize the superposition field in the region of avoidance, or wherein the multi-objective function is to minimize the superposition field in the region of interest and minimize the superposition field in the region of avoidance.

39. The method of claim 27, further comprising finding numerical solutions to the set of expressions to determine the discrete points of the Lagrangian function corresponding to the maximum, the minimum, or the none value.

40. The method of claim 27, wherein a bordered Hessian matrix is determined and minors of the bordered Hessian matrix and Karush-Kuhn-Tucker (KKT) conditions are used to determine if the determined discrete points are the maximum, the minimum, or the none value.

41. A system controlling targeted neural stimulation to a subject for treatment, the system comprising:
one or more processors; and
one or more memories having stored thereon computer-executable instructions that, when executed, cause the computing system to:
determine, at one or more processors, a stimulation signal for each of a plurality of electrodes positioned for affecting a region of interest and for affecting a region of avoidance, using a discretized point model representative of the region of interest and of the region of avoidance;
determine, at the one or more processors, a stimulation superposition signal using the stimulation signals for each electrode, the stimulation superposition signal being determined for each discretized point in the region of interest and in the region of avoidance and constraining all current fractions of the plurality of electrodes according to a maximum bound and/or a minimum bound;
from the determined stimulation signal superposition, perform, at the one or more processors, a multi-objective optimization on the stimulation signal data to maximize a superposition field at the region of interest and to minimize a superposition field at the region of avoidance according to a multi-objective function, wherein the instructions to perform the optimization include instructions to,
apply a smooth maximum operator to the objective function to form a differentiable objective function,
apply a Lagrange multiplier to obtain the set of expressions that maximizes the objective function and satisfies the constraints on the current fractions,
obtain from the set of expressions, discrete points of the Lagrangian function corresponding to a maximum, a minimum, or a none value,
use an epsilon-constraint method to construct a Pareto front of solutions for the multi-objective function,
select an objective function of the multi-objective function as a primary objective and converting the other objective functions to inequality constraints,
apply a generalized Lagrange multiplier to obtain the set of expressions that satisfies the multi-objective function and satisfies the constraints on the current fractions; and
identify, from the optimization, a stimulation signal combination for controlling each of the plurality of electrodes for treatment of the subject and corresponding to the optimization of the superposition field according to the objective function; and
provide control signals to the plurality of electrodes and based on the stimulation signal parameters to affect treatment of the region of interest and of the region of avoidance by the plurality of electrodes.

42. The computing system of claim 41, wherein the stimulation signal for each electrode is obtained using numerical methods or analytic methods.

43. The computing system of claim 41, wherein the discretized point model representative contains a single discretized point or a line of discretized points.

44. The computing system of claim 41, wherein the discretized point model contains different types of discretized points for the region of interest compared to the region of avoidance, wherein the different types of discretized points are selected from the group consisting of a single discretized point, a line of discretized points, a 2D area of discretized points, and volume of discretized points.

45. The computing system of claim 41, wherein the discretized point model contains a 2D area of discretized points.

46. The computing system of claim 41, wherein the discretized point model contains a volume of discretized points.

47. The computing system of claim 41, wherein the discretized point model is a finite element method model.

48. The computing system of claim 41, wherein the current fractions of all electrodes are simultaneously constrained to the maximum bound and the minimum bound during determination of stimulation superposition signal.

49. The computing system of claim 41, wherein the sum of all current fractions is constrained to be zero.

50. The computing system of claim 41, wherein the multi-objective function is to maximize the superposition field in the region of interest and to minimize the superposition field in the region of avoidance.

51. The computing system of claim 41, wherein the objective function is a superposition of an electric potential field at the region of interest, a first order spatial derivative of the electric potential field at the region of interest, a second order spatial derivative of the electric potential field at the region of interest, or a directional field or vector fields maximized or minimized in principal directions or along trajectories in the region of interest.

52. The computing system of claim 41, wherein the multi-objective function is to maximize the superposition field in the region of interest and maximize the superposition field in the region of avoidance, or wherein the multi-objective function is to minimize the superposition field in the region of interest and minimize the superposition field in the region of avoidance.

53. The computing system of claim 41, wherein instructions that, when executed, cause the computing system to find numerical solutions to the set of expressions to determine the discrete points of the Lagrangian function corresponding to the maximum, the minimum, or the none value.

54. The computing system of claim 41, wherein a bordered Hessian matrix is determined and minors of the bordered Hessian matrix and Karush-Kuhn-Tucker (KKT) conditions are used to determine if the determined discrete points are the maximum, the minimum, or the none value.

55. A non-transitory computer-readable storage medium storing executable instructions that, when executed by a processor, cause a computer to:
determine, at one or more processors, a stimulation signal for each of a plurality of electrodes positioned for affecting a region of interest and for affecting a region of avoidance, using a discretized point model representative of the region of interest and of the region of avoidance;
determine, at the one or more processors, a stimulation superposition signal using the stimulation signals for each electrode, the stimulation superposition signal being determined for each discretized point in the region of interest and in the region of avoidance and constraining all current fractions of the plurality of electrodes according to a maximum bound and/or a minimum bound;
from the determined stimulation signal superposition, perform, at the one or more processors, a multi-objective optimization on the stimulation signal data to maximize a superposition field at the region of interest and to minimize a superposition field at the region of avoidance according to a multi-objective function, wherein the instructions to perform the optimization include instructions to,
apply a smooth maximum operator to the objective function to form a differentiable objective function,
apply a Lagrange multiplier to obtain the set of expressions that maximizes the objective function and satisfies the constraints on the current fractions,
obtain from the set of expressions, discrete points of the Lagrangian function corresponding to a maximum, a minimum, or a none value,
use an epsilon-constraint method to construct a Pareto front of solutions for the multi-objective function,
select an objective function of the multi-objective function as a primary objective and converting the other objective functions to inequality constraints,
apply a generalized Lagrange multiplier to obtain the set of expressions that satisfies the multi-objective function and satisfies the constraints on the current fractions; and
identify, from the optimization, a stimulation signal combination for controlling each of the plurality of electrodes for treatment of the subject and corresponding to the optimization of the superposition field according to the objective function; and
provide control signals to the plurality of electrodes and based on the stimulation signal parameters to affect treatment of the region of interest and of the region of avoidance by the plurality of electrodes.
